(19) United States
(12) Patent Application Publication
(10) Pub. No.: US 2014/0212369 A1
(43) Pub. Date: Jul. 31, 2014

(54) SYSTEM, METHOD AND COMPOSITION FOR SKIN CARE TREATMENT

(71) Applicant: Suzanne Grayson, San Juan Capistrano, CA (US)

(72) Inventor: Suzanne Grayson, San Juan Capistrano, CA (US)

(21) Appl. No.: 14/103,897

(22) Filed: Dec. 12, 2013

Related U.S. Application Data
(60) Provisional application No. 61/757,432, filed on Jan. 28, 2013.

(51) Int. Cl. A61K 8/97; A61Q 19/08; A61K 8/67; A61K 8/60; A61K 8/41; A61K 8/49; A61K 8/368; A61K 8/34; A61K 8/64; A61K 8/55; A61K 8/43; A61K 8/81; A61K 8/37; A61K 8/92; A61K 8/891; A61Q 19/00

(52) U.S. Cl. CPC A61K 8/97; A61Q 19/00; A61Q 19/08; A61K 8/671; A61K 8/60; A61K 8/416; A61K 8/4926; A61K 8/368; A61K 8/602; A61K 8/345; A61K 8/347; A61K 8/64; A61K 8/4913; A61K 8/553; A61K 8/43; A61K 8/8152; A61K 8/37; A61K 8/922; A61K 8/891
USPC 424/78.03

(57) ABSTRACT

A cyclical dosage topical administration skin care one week's treatment of continuity complex active components and active booster serum active components effects anti-aging characteristics without adverse effects, caused by increasing dosage as shown when active ingredients are increased.

### PRIOR RELATED APPLICATION

This application claims priority to provisional application Ser. No. 61/757,432, filed Jan. 28, 2013, and incorporates said prior application herein in its entirety by reference thereto.

### BACKGROUND OF THE INVENTION

1. Field of the Invention

This invention relates to skin care. More specifically, this invention relates to a system, method and composition for skin care treatment, particularly with respect to reducing the appearance of aging skin.

2. Background and Discussion of Prior Art

Skin ages naturally with the passing of the years. Aging is accelerated by the action of the sun, which process is known as photo-aging and also by exposure to wind, salt and the like. With aging, skin acquires or exhibits any of a variety of problems including lines, wrinkles, lack of firmness and elasticity, a rough, non-homogeneous stratum corneum, age spots and actinic keratosis.

Although convenient for consumer use, cosmetic formulations of so-called skin-renewal compositions do not adequately meet the needs of the marketplace for aging skin treatments because of the necessary frequency and, greater potency or efficacy because the skin acclimates to potency levels, and yet such greater potency is hard to obtain without increased irritation or other undesirable side effects. Greater potency requires the use of very high concentrations of compositions which can be quite irritating and may even require professional supervision for safe use.

U.S. Pat. No. 5,382,432, granted Jan. 17, 1995 to McCook et al., and U.S. Pat. No. 5,401,517, granted Mar. 28, 1995 to Meyers et al., disclose cosmetic methods for skin treatment whereby in daily treatments progressively higher levels of the active component were provided to overcome the skin acclimation/irritation effects, and in order to provide the efficacious treatment over a several day regimen. However, such continued increases in dosages of the active chemical components caused adverse effects and was commercially undesired.

The present invention provides a seven day skin care treatment without the need for continuous increases in the dosages of active chemical or components over the several day treatment regimen.

The present invention is counterintuitive to McCook et al. and Meyers et al.

### SUMMARY OF THE INVENTION

The present invention, in one principal aspect, is a daily seven day regimen of efficacious skin treatment compositions without the need for continuing increases in the dosage levels, and yet provides an efficacious treatment regimen.

The present invention, as aforesaid, provides consistent levels of ingredients for daily Continuity Complex (CC) efficacy or benefits components, while the Active-Booster Serum (ABS) part of each daily formula includes entirely different compositions or ingredients each day. There is consequentially provided the novel Rotation Skin Therapy that eliminates the acclimation effect. In one aspect, there are modest increases in levels of only a few of the ingredients in the Continuity Complex (CC). The specific formulae further defined herein efficaciously provide the anti-aging effect while avoiding increased chemical damage and the skin acclimation/irritation effect.

### BRIEF DESCRIPTION OF THE FIGURES

FIG. 1 is Table I showing the Total Benefits for ABS components for a week's regimen;

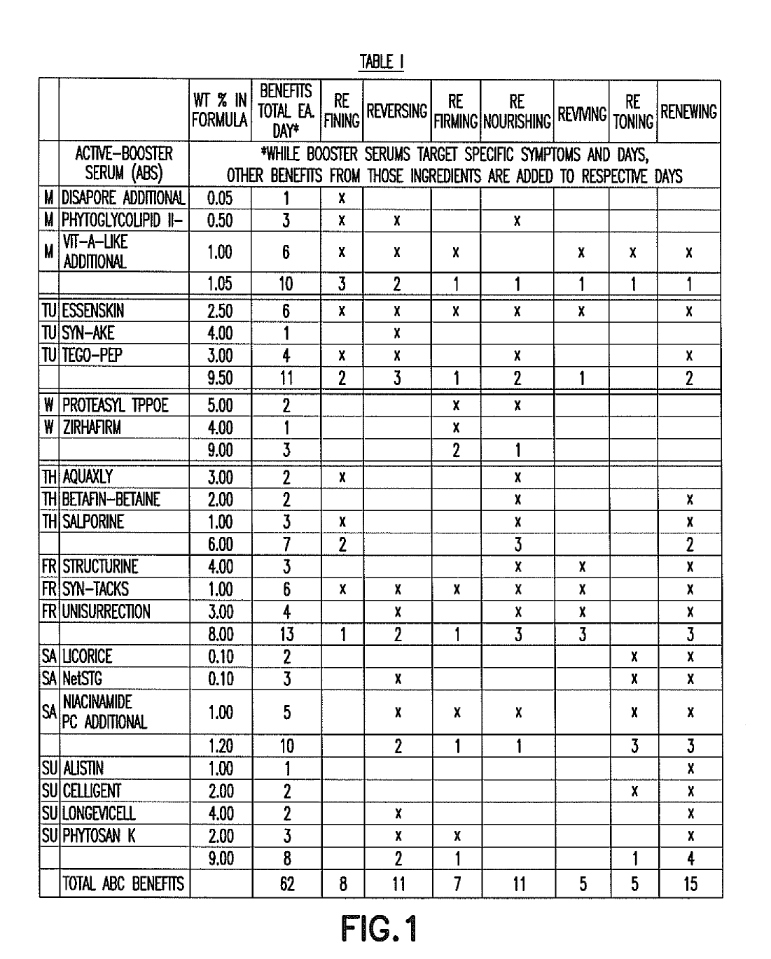

FIG. 2 is Table II showing the Total Benefits for the Continuity Complex (CC) components in a weekly regimen;

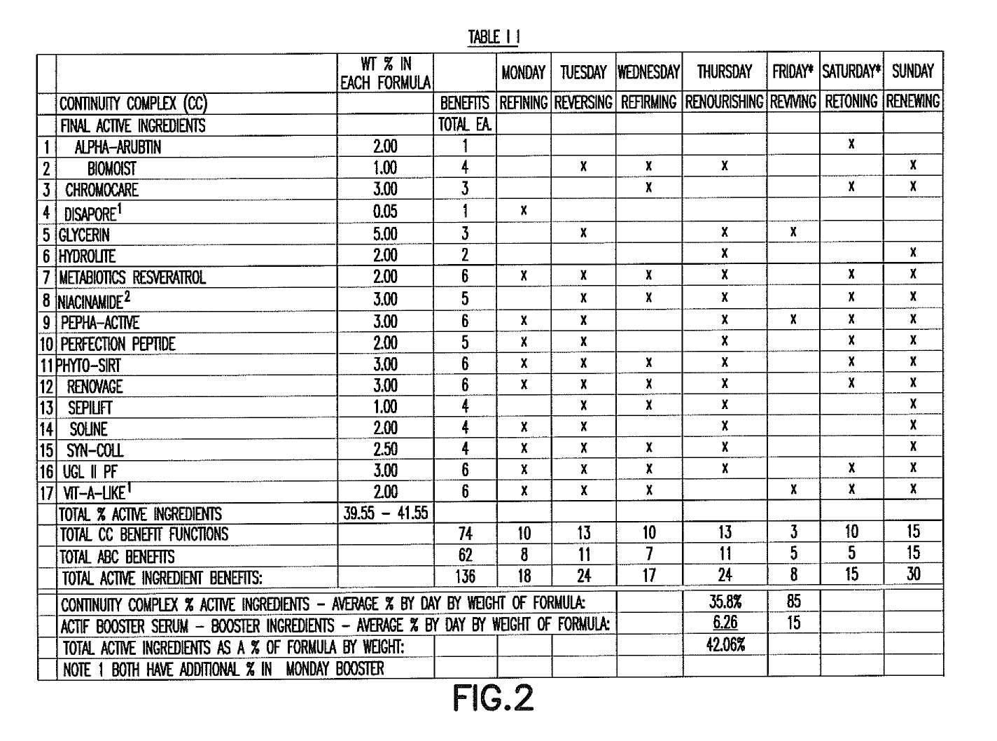

FIG. 3 is a Table III of the ingredient declaration showing diverse ingredients for the Monday-Refining regimen;

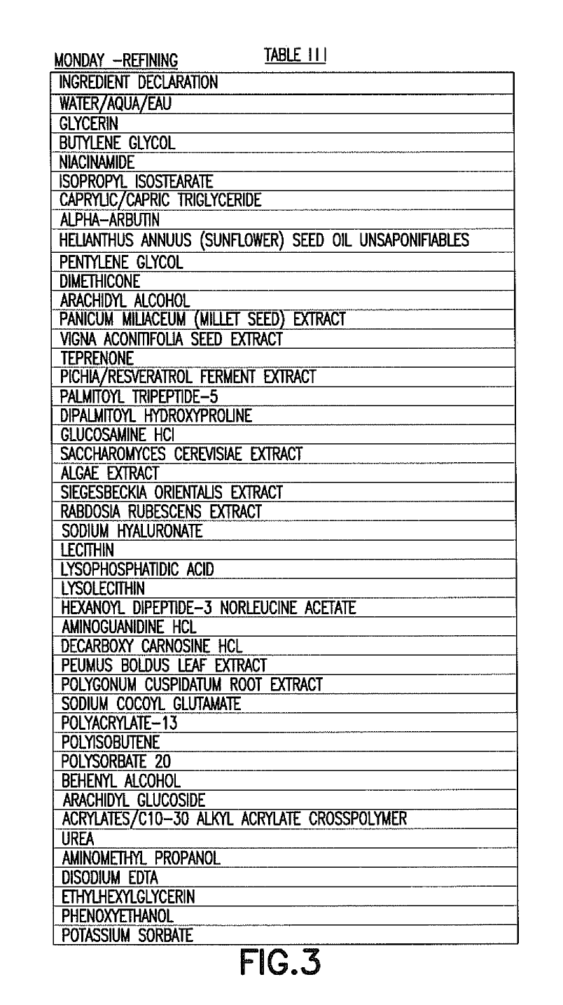

FIG. 4 is Table IV of the weight percentages for the Tuesday-Reversing components;

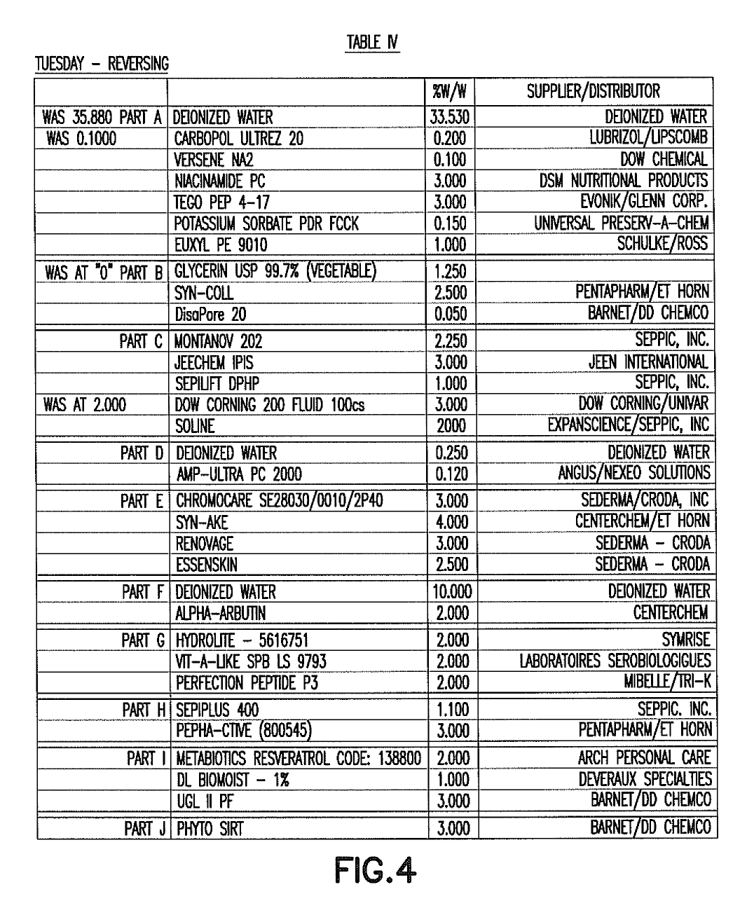

FIG. 5 is Table V of the weight percentages for the Wednesday-Refirming components;

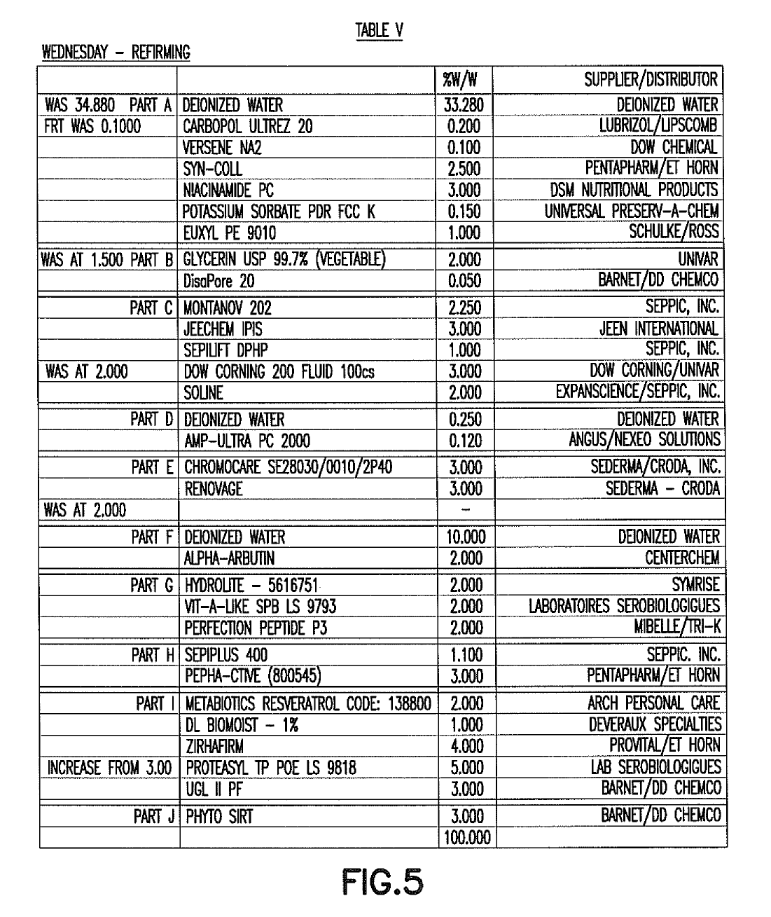

FIG. 6 is Table VI of the weight percentages for the Thursday-Renourishing components;

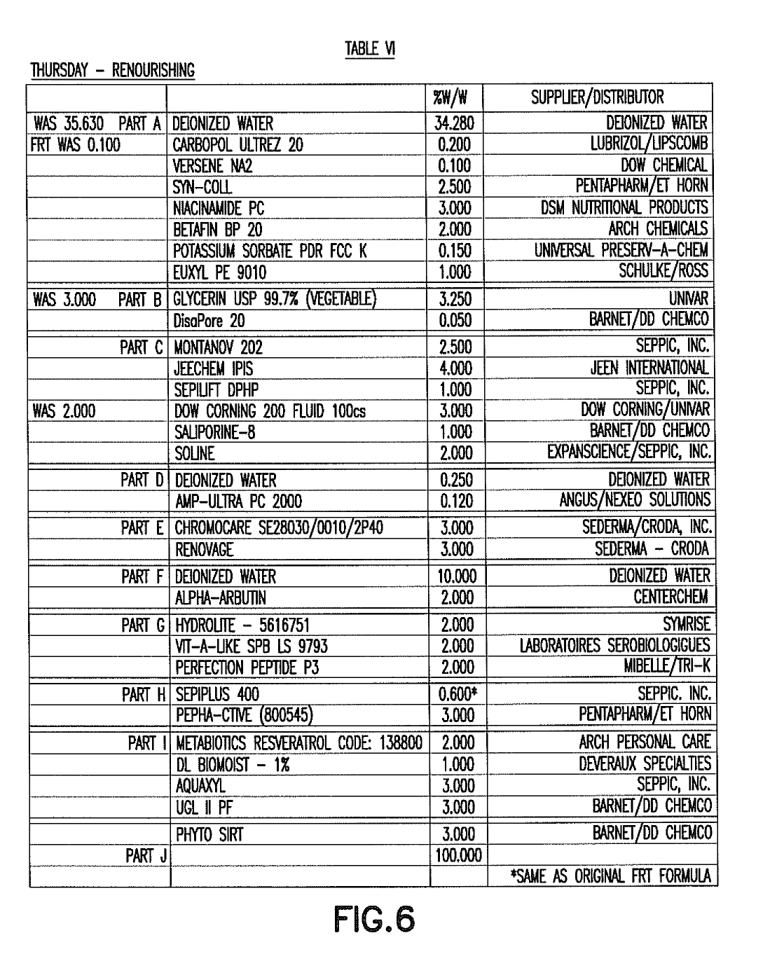

FIG. 7 is Table VII and the weight percentages for the Friday-Reviving components;

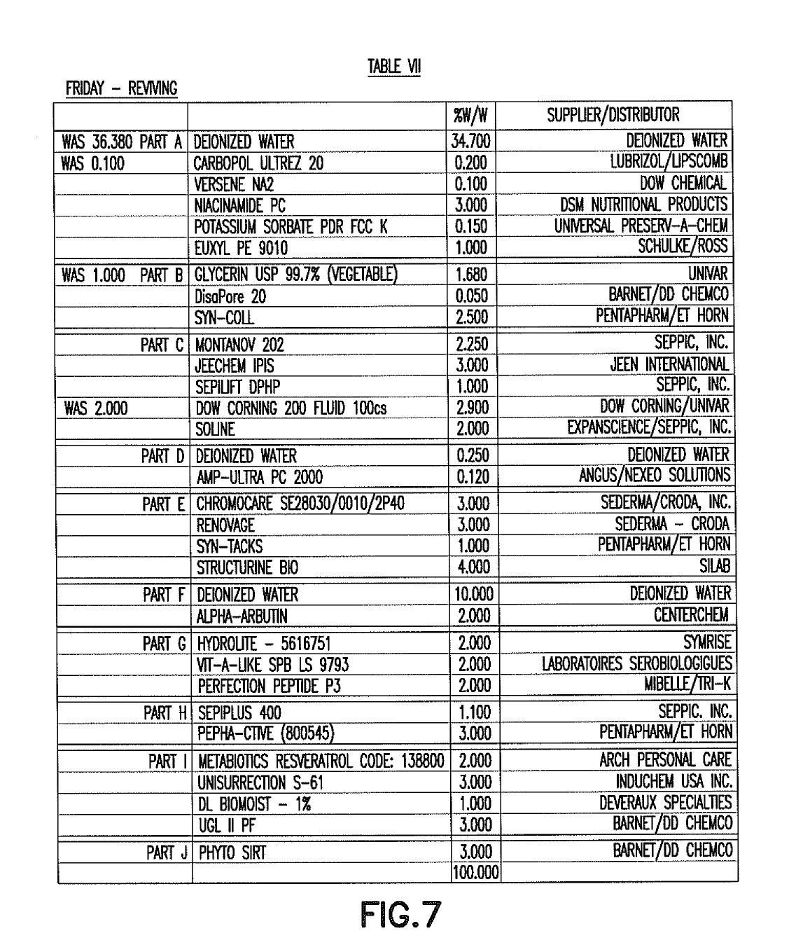

FIG. 8 is Table VIII and the weight percentages for the Saturday-Retoning components;

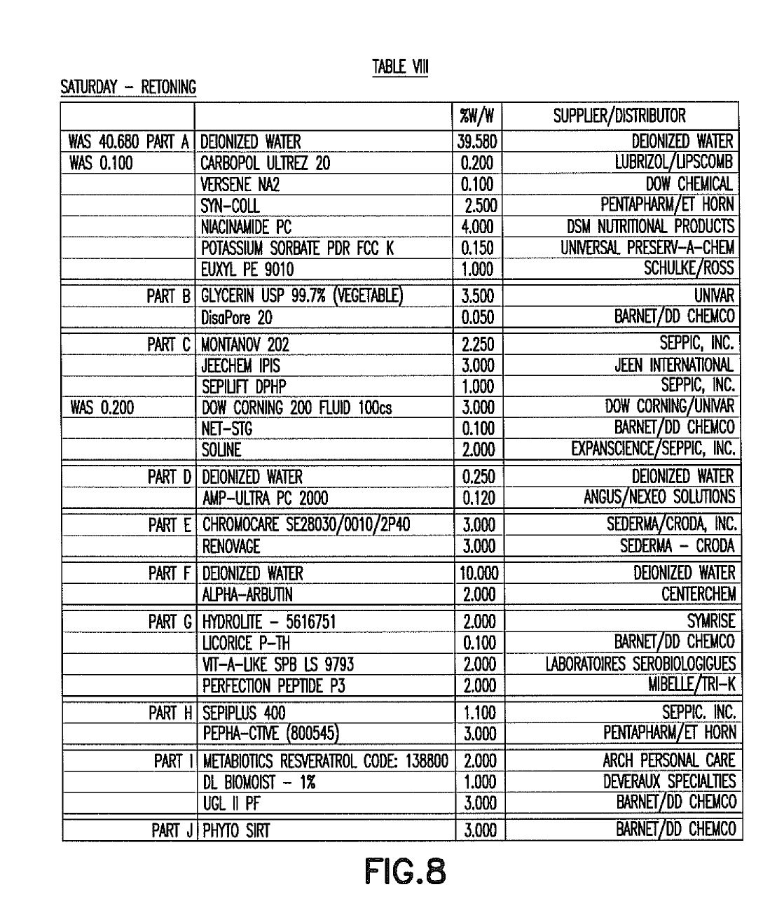

FIG. 9 is Table IX of the Sunday-Renewing ingredients or components;

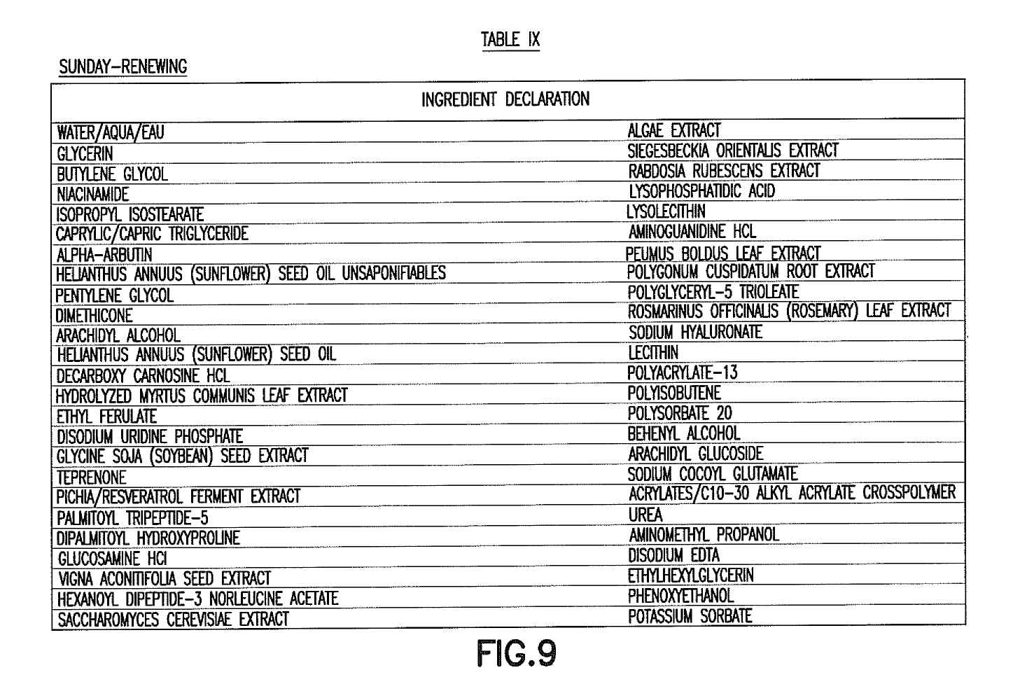

FIG. 10 is Table X showing the week 12 results of a subject self-assessment study;

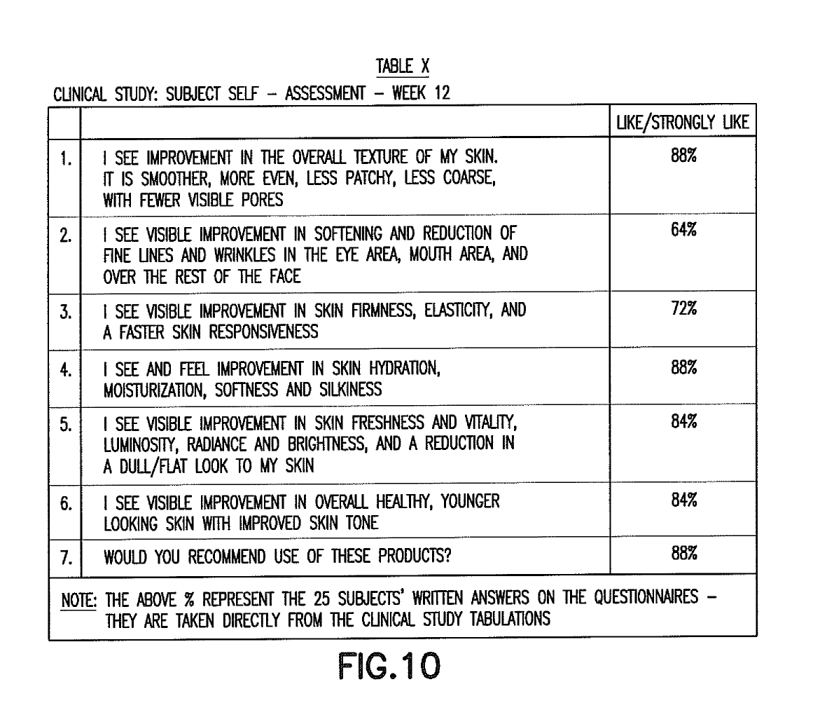

FIG. 11 is Table XI showing all rotation skin therapy actions and benefits; and

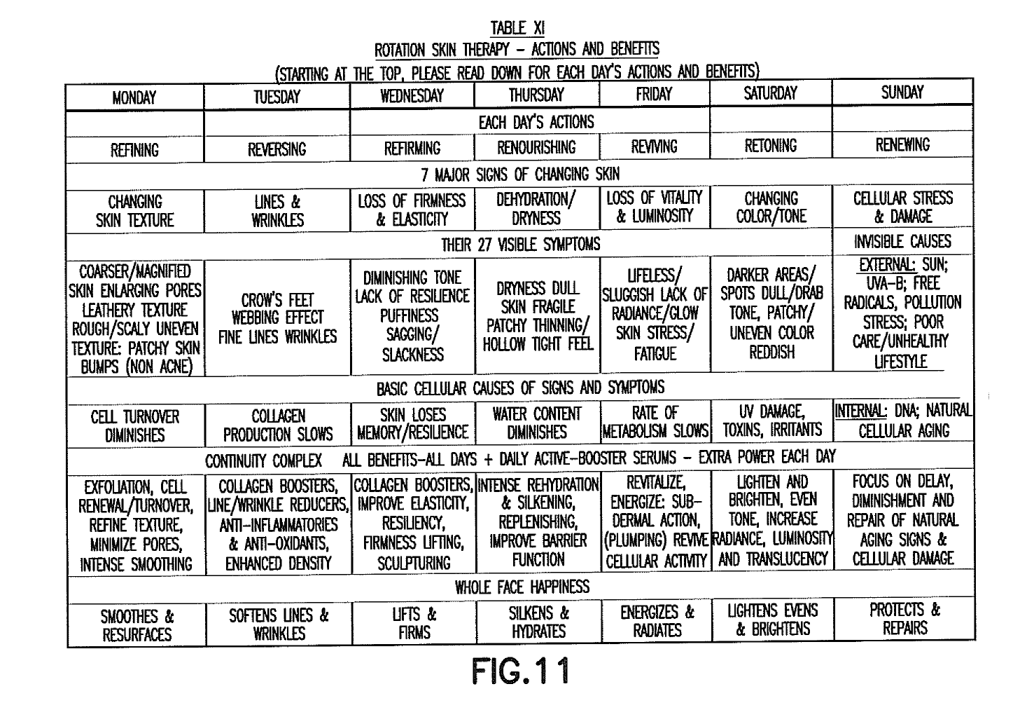

FIG. 12 is Table XII showing the rotation skin therapy with resultant actions and benefits.

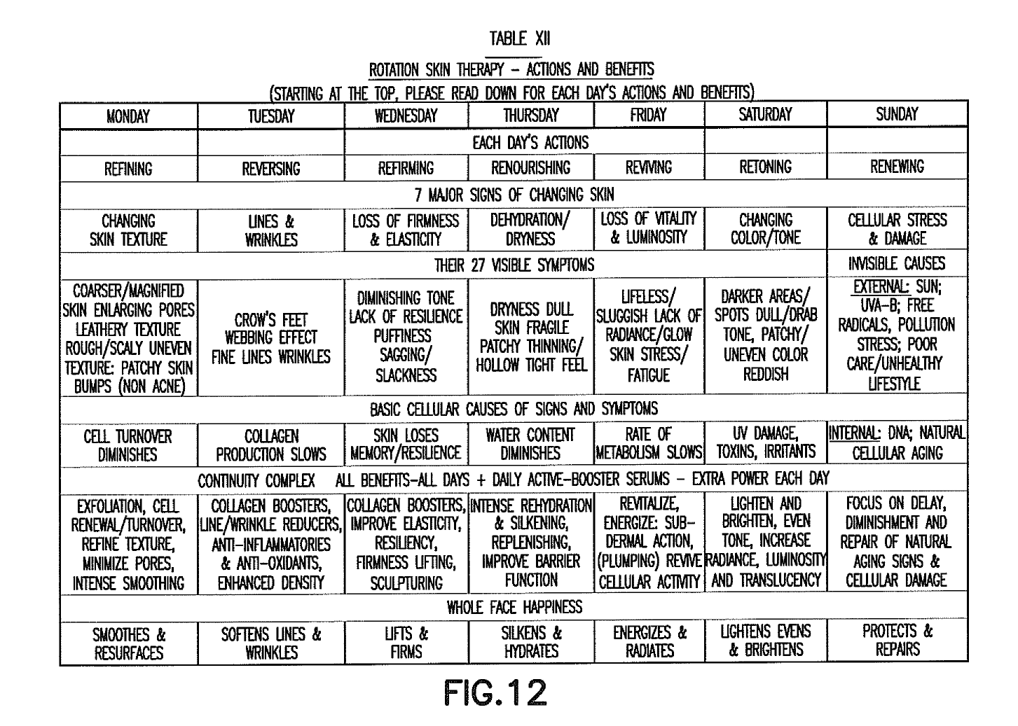

### DESCRIPTION OF THE INVENTION

An administration of skin treatment composition is undertaken wherein CC and ABS cyclical levels are rotated over the course of a several day regimen. The ABS rotating/cyclical and CC consistent levels thereby provide varying levels of refining, reversing, firming or refirming, renourishing, reviving, retoning and renewing effects over the several day regimens. The following Table I, II and Table XII demonstrate the system method and compositions of the present invention. The attached Tables III-XI set out the specific chemicals, compositions or components useful in a Monday through Sunday treatment.

Referring specifically to Table I, certain ABS components, e.g. phytoglycolipd II, is only in the Monday composition, yet it has benefits that contribute to other day's benefits as shown.

The Active Booster Serum (ABS) targets each specific day's aging system.

The attached Tables III, IV, V, VI, VII VIII and IX describe the ingredients for, respectfully Monday—Refining, Tuesday—Reversing, Wednesday—Refirming, Thursday—Renourishing, Friday—Reviving, Saturday—Retoning and Sunday—Renewing.

A week's regimen includes 7 different specifically targeted anti-aging treatments one for each day of the week, as exemplified:

Monday—Refining for coarsening pores and patchy features;

Tuesday—Reversing the appearance of and wrinkles;

Wednesday—Refirming for elasticity and reconditioning;

Thursday—Renourishing for dry, patchy and thinning skin;

Friday—Reviving for lifeless, fatigued, flat and damaged skin;

Saturday—Retoning for dull, uneven darkening and reddening skin; and

Sunday—Renewing for stressed and damaged skin.

Each formula as set out in Tables I and II is, in a preferred embodiment, topically applied to the face and around the eyes twice daily.

The Continuing Complex (CC) compositions target the major signs of aging, including slowdown in natural exfoliation which results in one or more symptoms, namely, magnified/coarsening skin texture, enlarged pores, rough, dull and/or uneven texture, dry patches, and the like.

A self-assessment clinical study of 25 subjects who underwent an extended clinical regimen of the foregoing treatment supervised by a clinical dermatologist is reported in FIG. X.

A one week regimen as afore-described provides several benefits for the user, by way of example.
1. After one weeks' use, because of the total of CC and ABS ingredients, the user in effect receives the benefits of higher concentrations of more actives, (35), which have been accumulated to address all twenty-seven of the signs of aging skin than is possible to obtain with any single facial “anti-aging” product used over the same period of time, no matter the retail price. The reason for this achievement is that it overcomes the limitation of 60-70% by weight water required for typical formulations, which does not allow for this many clinically tested levels of active ingredients (35), plus the inactive ingredients in a single formulation.
2. The potential for skin irritation with use of higher concentrations of actives is lessened considerably in as much as the user is not using the same product every day.
3. It is expected that the user's skin benefits will increase over time because her skin will not “plateau” in benefits as it becomes accustomed to the same product every day, as is often the case with the regular use of a prior art single product.
4. An all-in-one “anti-aging” program for face and throat. No other moisturizer or facial “anti-aging” product is necessary including the area surrounding the eyes, generally the sign of tiny lines. It is common in the prior art to provide separate compositions for facial and around the eyes treatments, as well as additional specific purpose serums.

It is understood that while the invention has described as hereinabove and attached, various modifications and changes will be apparent to one skilled in the art within the scope of the invention, that the invention is therefore to be reasonably defined by the adjoined claims.

What is claimed is:

1. A method for treating skin comprising:
topically applying CC and ABS components on the skin in rotation to provide varying levels of refining, reversing, refirming, renourishing, reviving, retoning, and renewing effects, wherein each day of the week, a different set of one or more ABS components are applied to the skin depending upon the day, and a plurality of CC components are applied seven days of the week.

2. The method of claim 1, wherein the skin treating effects comprise anti-aging effects.

3. The method of claim 2, wherein the anti-aging skin treating comprises a weekly regimen of twice-a-day topical applications further comprising;
topically applying in day one, compositions effecting refining of the skin;
topically applying in day two, compositions effecting reversing of wrinkling;
topically applying in day three, compositions effecting firming of the skin;
topically applying in day four, compositions effecting renourishing of the skin moisturizing ability;
topically applying in day five, compositions effecting reviving of the skin;
topically applying in day six, compositions effecting retoning of the skin; and
topically applying in day seven, compositions effecting renewing of the skin.

4. The method of claim 1, wherein at least one ABS component for Monday-refining comprises a phytoglycolipid.

5. The method of claim 4, wherein the phytoglycolipid ABS component is topically applied on the first day of the week.

6. The method of claim 5, wherein the phytoglycolipid ABS component is only applied on the first day of the week, and wherein the first day skin benefit also effects the other days skin benefits.

7. The method of claim 1, further comprising topically applying the ABS and CC components twice daily.

8. The method of claim 1, wherein the ABS and CC components comprise different compositions.

9. The method of claim 1, wherein the ABS component comprises topically applying the phytoglycolipid on the first day only; and wherein its first day skin benefit also effects the other days skin benefits.

10. The method of claim 9, wherein the anti-aging skin treating comprises a weekly regimen of twice-a-day topical applications further comprising;
topically applying in day one, compositions effecting refining of the skin;
topically applying in day two, compositions effecting reversing of wrinkling;
topically applying in day three, compositions effecting firming of the skin;
topically applying in day four, compositions effecting renourishing of the skin moisturizing ability;
topically applying in day five, compositions effecting reviving of the skin;
topically applying in day six, compositions effecting retoning of the skin; and
topically applying in day seven, compositions effecting renewing of the skin.

11. The method of claim 1, wherein the plurality of CC components comprises 17 CC components.

12. The method of claim 1, wherein the topically applying CC and ABS compositions comprises Rotation Skin Therapy.
